(19) United States
(12) Patent Application Publication
(10) Pub. No.: US 2004/0102698 A1
(43) Pub. Date: May 27, 2004

(54) Patient positioning system for radiotherapy/radiosurgery based on magnetically tracking an implant

(76) Inventors: Stefan Vilsmeier, Kufstein (AT); Stephan Frohlich, Aschhem (DE); Stephan Erbel, Munchen (DE)

Correspondence Address: RENNER, OTTO, BOISSELLE & SKLAR, LLP, Nineteenth Floor, 1621 Euclid Avenue, Cleveland, OH, 44115-2191, US

(21) Appl. No.: 10/634,133

(22) Filed: Aug. 4, 2003

Related U.S. Application Data
(60) Provisional application No. 60/464,247, filed on Apr. 21, 2003.

(30) Foreign Application Priority Data
Aug. 8, 2002 (EP) 02 017 736.6

(51) Int. Cl. A61B 5/05

(52) U.S. Cl. 600/424

(57) ABSTRACT

A method for positioning a patient or detecting a target volume in radiotherapy or radiosurgery includes positionally referencing at least one implant in the vicinity of the target volume and inductively stimulating the at least one implant. Emission from the at least one inductively stimulated implant is detected and a position of the at least one implant is determined based on the detected emission. The current position of the target volume is determined based on the determined position of the at least one implant. Further diagnostic, two-dimensional or three-dimensional image data sets can be recorded in accordance with breathing.

### RELATED APPLICATION DATA

This application claims priority of U.S. Provisional Application No. 60/464,247, filed on Apr. 21, 2003, which is incorporated herein by reference in its entirety.

### FIELD OF THE INVENTION

The present invention relates to a method and a system for exactly positioning a patient in radiotherapy and/or radiosurgery and/or for taking into account shifts in internal structures of the patient caused by breathing, both during the actual treatment and while recording the image data necessary for said treatment.

### BACKGROUND OF THE INVENTION

In radiotherapy and radiosurgery, major progress has been achieved in recent times in dosage planning. The aim is to use higher radiation doses that are applied to a target volume, for example, a tumor, as precisely as possible, without damaging the surrounding regions. Although dosage planning has been shown to be relatively successful, the use of higher doses, which in certain cases are even administered in a single or in a few fractions, is hindered by the fact that the patient or the section of the body to be treated can only be positioned relatively imprecisely. In order to avoid major damage to healthy tissue, exact positioning is essential.

Even optimally positioning the patient can, in certain cases, be insufficient, if the target tissue, which is to be irradiated, moves within the patient. Breathing movement can result in a shift of up to about 2 cm. In order to be able to ensure optimal treatment even in these cases, it is necessary to know the exact position of the target area within the patient at any time during the treatment. If this position is known, then it is possible to activate the therapy device only when the target volume in the patient is within a range of tolerance about the target area of the therapy device or, if it can be stipulated in the design of the machine in question, to slave the target area of the therapy machine to the movement of the target.

A related problem arises when recording the diagnostic images on which the treatments are based. If the interior of the patient moves while the image data is being recorded, then artifacts result in the images and the geometrical proportions of the interior of the patient are distorted. Correspondingly, the target volume within the patient is not detected in its true size, but may be shown too large or too small.

Furthermore, established x-ray-based methods generally only detect the osseous structures of the patient and are thus relatively unsuitable for treating organs that can shift relative to these osseous structures (for example, the prostate). A further disadvantage of x-ray-based methods is their limited resolution in time, and the resultant radiation load, when tracking shifts caused by breathing over a long period of time. The cost of stereoscopic x-ray systems is an obstacle for many applications.

### SUMMARY OF THE INVENTION

The present invention may include any of the features cited in the claims and in this description, individually or in any combination.

In accordance with one aspect of the invention, the invention is directed to a method for positioning a patient or detecting a target volume in radiotherapy or radiosurgery. The method can include positionally referencing at least one implant in the vicinity of the target volume and inductively stimulating the at least one implant. Emission from the at least one inductively stimulated implant is detected and a position of the at least one implant is determined based on the detected emission. The current position of the target volume is determined based on the determined position of the at least one implant.

In accordance with another aspect of the invention, the invention is directed to a method for recording diagnostic, two-dimensional or three-dimensional image data sets in accordance with breathing. The method can include introducing at least one implant into the patient in the vicinity of the target volume and inductively stimulating the at least one implant. Emission from the at least one inductively stimulated implant is detected and a position of the at least one implant is determined based on the detected emission. Image data is recorded based on the position of the at least one implant.

### BRIEF DESCRIPTION OF THE DRAWINGS

These and further features of the present invention will be apparent with reference to the following description and drawings, wherein:

FIG. 1 is a flow chart illustrating a method of positioning a patient or detecting a target volume in accordance with the present invention.

### DETAILED DESCRIPTION OF THE INVENTION

In the detailed description that follows, corresponding components have been given the same reference numerals regardless of whether they are shown in different embodiments of the present invention. To illustrate the present invention in a clear and concise manner, the drawings may not necessarily be to scale and certain features may be shown in somewhat schematic form.

In one embodiment, the present invention employs one or more implants whose position can be determined via a magnetic tracking system. The implants are supplied with energy, for example, by inductively transferring electromagnetic energy via a dynamic field, which is beamed into the patient from without. In this way, cabling for the implant and an energy storage device in the implant can be omitted. It is to be appreciated that omitting external cabling significantly reduces the risk of infection for the patient.

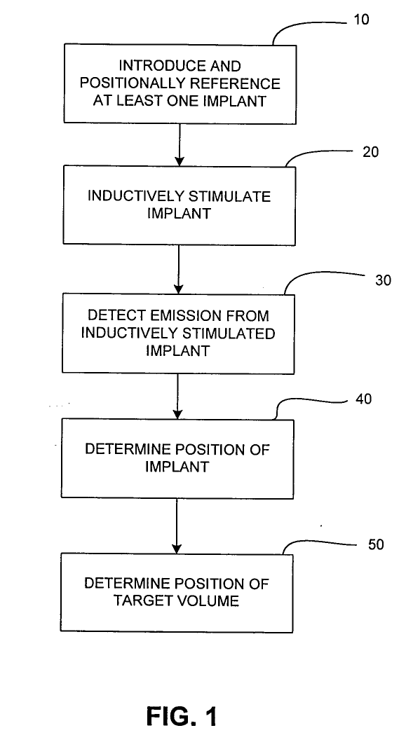

With reference to FIG. 1, a method for positioning a patient or detecting a target volume is illustrated. The method can include introducing and positionally referencing 10 at least one implant in the vicinity of the target volume and inductively stimulating 20 the at least one implant. Emission from the at least one inductively stimulated implant can be detected 30 and a position of the at least one implant can be determined 40 based on the detected emission. The current position of the target volume can be determined 50 based on the determined position of the at least one implant. While the present invention is being discussed in the context of radiotherapy and/or radiosurgery applications, it is to be appreciated that the present invention is amenable to other applications.

### Magnet Tracking, Using Implants for Positioning, on the Treatment Device

The electromagnetic energy absorbed can be at least partially re-emitted by the implant, preferably in the form of an alternating field. If the signal emitted by the implant is measured in the vicinity of the patient, then the position of the implant relative to the measuring device or devices can be determined. It is to be appreciated that the position of the emitter is determined using a magnetic tracking system in a suitable manner.

If the position of the measuring points (i.e., the position of the points at which the emitted electromagnetic radiation is detected or measured), relative to the therapy device is known, then the position of the implant relative to the therapy device can therefore also be determined.

In accordance with one exemplary embodiment, the invention will now be illustrated using the example of a conventional linear accelerator whose gantry can rotate about a horizontal axis. However, it is to be appreciated that the present invention can equally be used with linear accelerators that are guided on robot arms having a number of degrees of freedom or also with therapy systems based on radioactive decay.

The position of the measuring points relative to the therapy device can be known either from their fixed connection to the therapy device or can be determined in real time via a six-dimensional tracking system (3 translatory and 3 rotatory degrees of freedom). In one embodiment, the ExacTrac™ system of BrainLAB AG may be employed as an example of a system capable of tracking the position of a measuring head, fitted with one or more sensors, in six dimensions. This second solution has the advantage that the sensors for measuring can be arranged very near to the patient, and then removed again after measuring. The nearer to the emitter the measurement is taken, the less output is required by the emitting implant and the less effect any interference fields have.

### Magnetic Tracking, Using Implants for Treatment Triggered by Breathing, on the Treatment Device

If the position of the implant can be determined several times a second, then any shift of the implant caused by breathing and, therefore, any shift of the target volume within the patient, can be determined. Knowledge of the position of the implant can be used during the treatment to activate the therapy device when the position of the target volume in the patient is within a predetermined range of tolerance about the current target point of the therapy device. Alternatively, the therapy device or the patient can also be shifted according to the movement of the target volume.

In cases in which there is a negative interaction between the therapy device and the magnetic tracking system, the same function can also be indirectly achieved by linking it to another tracking device (for example, ExacTrac™ of the firm BrainLAB AG, Kirchheim/Heimstetten). In addition, at the same time as the measurement is taken magnetically, a physiological change in the patient is measured, for example, the movement of the thorax and the stomach wall during breathing. By correlating these two signals, it is possible, even at a late stage, to control the subsequent irradiation exclusively on the basis of the signal from the second tracking system.

### Magnetic Tracking, Using Implants for Recording Diagnostic Images, Triggered by Breathing

Recording the diagnostic images on which the treatments are based can be solved similarly. In order to avoid artifacts in the images and distortion of the geometrical proportions in the data sets, it is necessary for all the image data to match one lung filling of the patient as exactly as possible. If all the tomographic images are taken at times when the patient has the same lung filling, then it is ensured that each point in the patient's interior is only imaged in exactly one tomographic image. In order to achieve this, the position of the implant can be continuously tracked on the imaging device (e.g., CT), and recording a tomographic image is only started when the position of the implant in the patient is within a predetermined range of tolerance about a previously established position.

### Magnetic Tracking, Using Implants for Preparing for Positioning, Outside the Treatment Position

Interference fields, and metallic and other electrically conducting objects, which distort magnetic fields, are critical for magnetic tracking. If, despite all measures, it is not possible to measure to a sufficient accuracy in the vicinity of the therapy machine, then the present invention provides an alternative embodiment.

By combining the magnetic tracking system with another, e.g. optical, 3D tracking system that can spatially determine the position of the measuring points, it is possible to measure the position of the implant in a space or in a region of a space in which there are fewer sources of interference. A possible implementation of this approach will now be explained by way of the ExacTrac™ system of BrainLAB AG (Kirchheim/Heimstetten). In this embodiment, the electromagnetic measuring points are in a known, preferably fixed, spatial relation to reference points that are detected by the tracking system. Such tracking systems include those disclosed in commonly owned U.S. Pat. No. 6,351,659, which is incorporated herein by reference in its entirety. In this embodiment, reflective spheres are situated on a solid structure, which is fixedly attached to the patient or to the couch on which the patient is lying. The position of the implant relative to the measuring points can then be determined and the position of the implant relative to the reflecting spheres can be determined using the known spatial relation between the reflecting spheres and said measuring points.

If the patient is then moved to the therapy device, this spatial relationship should not changed. This can be realized by moving the patient, together with the couch on which he is laid, to the therapy device. Additionally, the patient can also be fixed to said couch.

When the patient is lying on the patient table of the therapy machine, the position of the reflective spheres is detected in real time. Since the spatial relationships between the implant and the target volume in the patient and between the implant and the reflective spheres are known, the patient can then be positioned on the basis of the reflective spheres such that the target volume in the patient is positioned correctly relative to the therapy machine.

### Magnetic Tracking, Using Implants for Preparing for Treatment Triggered by Breathing, Outside the Treatment Position

In another embodiment, it may be desirable in treatments triggered by breathing to take the magnetic measurements away from the therapy machine. In addition to the advantage that interference fields and signals can be avoided, this can reduce the time that the patient spends on the therapy machine. As already described further above, a physiological change in the patient is additionally measured, for example, the movement of the thorax and the stomach wall during breathing, at the same time as the measurement is taken magnetically. By correlating these two signals, it is possible to establish a criterion for the independent system that ensures that the implant is situated in the correct position in the patient.

If the patient is subsequently moved to the therapy device, then he can be irradiated exclusively on the basis of the signal from the second tracking system, which likewise has to be present on the therapy device.

Although particular embodiments of the invention have been described in detail, it is understood that the invention is not limited correspondingly in scope, but includes all changes, modifications and equivalents coming within the spirit and terms of the claims appended hereto.

What is claimed is:

1. A method for detecting a target volume in radiotherapy or radiosurgery, said method comprising:
positionally referencing at least one implant in the vicinity of the target volume;
inductively stimulating the at least one implant;
detecting emission from the at least one inductively stimulated implant;
determining a position of the at least one implant based on the detected emission; and
determining the current position of the target volume based on the determined position of the at least one implant.

2. The method as set forth in claim 1, further comprising:
introducing the at least one implant into the patient in the vicinity of the target volume;
detecting the position of the at least one introduced implant using an imaging system before a radiation treatment;
referencing the at least one introduced implant relative to inner organs or other body structures.

3. The method as set forth in claim 2, further comprising:
after detecting the position of the at least one introduced implant, moving the patient to a therapy device;
at the therapy device, generating a dynamic electromagnetic field in the vicinity of but outside the patient, wherein the at least one implant inductively absorbs energy via the electromagnetic field and the at least one implant at least partially re-emits the absorbed energy in the form of a second electromagnetic signal;
detecting the second electromagnetic signal outside the patient; and
determining the position of the at least one implant relative to measuring points at which the second electromagnetic signal is detected, the position of said measuring points relative to the therapy device being known.

4. The method as set forth in claim 3, further comprising:
determining the current position of the target volume based on the determined position of the at least one implant and knowledge of the position of the patient's inner organs relative to the at least one implant.

5. The method as set forth in claim 4, further comprising:
shifting the patient such that the target volume can be captured by a therapy beam from the therapy device.

6. The method as set forth in claim 4, further comprising:
adjusting a therapy beam from the therapy device to the current position of the target volume.

7. The method as set forth in claim 4, further comprising:
continuously detecting the position of the at least one implant; and
based on the continuously detected position, determining a shift in the position of the target volume caused by breathing.

8. The method as set forth in claim 4, further comprising:
based on the current position of the at least one implant, activating the therapy device only when the position of the target volume is within a predetermined range about a current target point of the therapy device.

9. The method as set forth in claim 8, wherein knowledge of the current position of the target volume within the patient is used to adjust the therapy device such that the target point of the therapy device follows the shift of the target volume.

10. The method as set forth in claim 3, wherein the measuring points are situated on a rotating portion of a linear accelerator.

11. The method as set forth in claim 3, wherein the measuring points are integrated into a treatment couch of the therapy device.

12. The method as set forth in claim 3, wherein one or more measuring points are attached to a solid, mobile structure which position relative to the therapy device is tracked three-dimensionally by means of a real-time tracking system.

13. The method as set forth in claim 2, wherein the at least one implant includes one or more coils.

14. The method as set forth in claim 13, wherein the at least one implant includes a number of coils whose axes are not parallel to each other.

15. The method as set forth in claim 13, wherein the coils in the at least one implant are connected to different oscillating circuits having different resonance frequencies.

16. The method as set forth in claim 1, wherein:
while the at least one implant is tracked, the patient is situated in a space or region of a space in which there are as few interference fields as possible and in which there are as few metallic parts as possible;
the position of the at least one implant relative to measuring points is determined;
the measuring points are fixedly connected to the patient or to a couch on which the patient is lying;
the measuring points are fitted with a reference means which allows the position of the measuring points to be determined using an independent, three-dimensional tracking system;
after electromagnetic measuring, the patient is moved to a therapy device in such a way that the spatial relationship between the patient and the measuring points is not changed; and
the patient is positioned relative to the therapy device by way of the reference means.

17. The method as set forth in claim 16, wherein the independent, three-dimensional tracking system is an optical infrared camera system.

18. The method as set forth in claim 3, wherein:
at least one of the steps is performed in a space adjacent to a treatment position; and
a tracking system additionally tracks the movement and position of external infrared markings, wherein the position and movement of the implant is referenced with respect to the position and movement of the external markings, and wherein positioning, gating and/or beam tracking are based only on tracking the external markings.

19. A method for recording diagnostic, two-dimensional or three-dimensional image data sets in accordance with breathing, said method comprising:
introducing at least one implant into the patient in the vicinity of the target volume;
inductively stimulating the at least one implant;
detecting emission from the at least one inductively stimulated implant;
determining a position of the at least one implant based on the detected emission; and
recording image data based on the position of the at least one implant.

20. The method as set forth in claim 19, further comprising:
at the imaging system, generating a dynamic electromagnetic field in the vicinity of but outside of the patient, wherein the at least one implant inductively absorbs energy via the electromagnetic field and the at least one implant at least partially re-emits the absorbed energy in the form of an electromagnetic signal;
detecting the electromagnetic signal outside the patient;
determining the position and/or orientation of the at least one implant relative to measuring points at which the electromagnetic signal is detected, the position of said measuring points relative to the imaging system being known; and
based on knowledge of the position of the at least one introduced implant, causing the imaging system to record data only when the position of the implant is within a tolerance range within the patient.
